(19) United States
(12) Patent Application Publication
(10) Pub. No.: US 2021/0106792 A1
(43) Pub. Date: Apr. 15, 2021

(54) GUIDEWIRES AND RELATED METHODS AND SYSTEMS

(71) Applicant: Transmural Systems LLC, Andover, MA (US)

(72) Inventor: Nasser Rafiee, Andover, MA (US)

(21) Appl. No.: 17/129,699

(22) Filed: Dec. 21, 2020

Related U.S. Application Data
(63) Continuation of application No. PCT/US2019/038580, filed on Jun. 21, 2019.
(60) Provisional application No. 62/688,409, filed on Jun. 22, 2018.
(60) Provisional application No. 62/688,374, filed on Jun. 21, 2018.

(51) Int. Cl. A61M 25/09; A61L 31/02; A61B 18/14

(52) U.S. Cl. CPC A61M 25/09; A61L 31/022; A61B 18/1492; A61B 2018/00577; A61M 2025/09083; A61M 2025/09133; A61M 25/09041

(57) ABSTRACT

In accordance with some implementations, various embodiments of a guidewire or catheter having an elongate core wire are provided. The guidewire includes a core wire having a proximal end, a distal end and is defined by an outer surface between the proximal end and the distal end of the core wire. The core wire has a centerline that traverses the length of the core wire from the proximal end to the distal end of the core wire. The core wire includes a proximal region having a first cross sectional dimension and a distal region having a plurality of sections of different cross-sectional dimension that are smaller than the first cross-sectional dimension. The guidewire further includes a coil wrapped around a distal end portion of the core wire.

### CROSS-REFERENCE TO RELATED APPLICATIONS

The present patent application is a continuation of and claims the benefit of priority to International Application No. PCT/US2019/38580, filed Jun. 21, 2019, which in turn claims the benefit of priority to U.S. Patent Application Ser. No. 62/688,374 filed Jun. 21, 2018 and to U.S. Patent Application Ser. No. 62/688,409 filed Jun. 22, 2018. Each of the aforementioned patent applications is hereby incorporated by reference in its entirety for any purpose whatsoever.

### FIELD OF THE DISCLOSURE

The present disclosure relates to various embodiments of guidewires.

### BACKGROUND

Various embodiments of guidewires and catheters are known in the art. Some of these are steerable devices. The present disclosure improves on the state of the art.

### SUMMARY OF THE DISCLOSURE

The purpose and advantages of the present disclosure will be set forth in and become apparent from the description that follows. Additional advantages of the disclosed embodiments will be realized and attained by the methods and systems particularly pointed out in the written description hereof, as well as from the appended drawings.

In accordance with some implementations, various embodiments of a guidewire can include a core wire having a proximal end and a distal end. The core wire is defined by an outer surface between the proximal end and the distal end of the core wire. The core wire has a centerline that traverses the length of the core wire from the proximal end to the distal end of the core wire. The core wire includes a proximal region having a first cross sectional dimension and a distal region having a plurality of sections of different cross-sectional dimension that are smaller than the first cross-sectional dimension. The guidewire further includes a coil wrapped around a distal end portion of the core wire.

In some implementations, the distal region of the core wire can include at least three sections of different cross sectional dimension that are connected to each other by tapering transition regions. A most proximal of the tapering transition regions can be between about one and about two inches in length. A second most proximal of the tapering transition regions can be between about three and about eight inches in length. A third most proximal of the tapering transition regions can be between about one half and about one inch in length. In other implementations, the distal region of the core wire can include at least four sections of different cross sectional dimension that are connected to each other by tapering transition regions, and the lengths of the transition regions can be the same as or be similar to those set forth above.

In some embodiments, the distal end of the core wire can terminate in a section of constant diameter. If desired, the coil can traverses between about 3 cm and about 12 cm of the length of the core wire, or any increment therebetween of one millimeter. The coil can traverse between about 5 cm and about 10 cm of the length of the core wire, or any increment therebetween of one millimeter. In some embodiments, the core wire can include a cobalt chromium alloy. If desired, the coil can include an alloy of platinum and tungsten. In some embodiments, at least one of the core wire and the coil can be coated with a layer of lubricious material.

The system further provides embodiments of an electrosurgical system that includes an electrical power source and a guide wire as described herein. The electrical power source is configured to be selectively electrically coupled to the guide wire. Preferably, the coil is welded to the core wire to facilitate the delivery of electrical energy to a target tissue area. The guidewire is preferably coated along a majority of its length with an electrically insulating material. Preferably, a proximal region of the core wire is exposed and not covered by the electrically insulating material. The proximal region of the core wire that is exposed can includes a roughened surface formed by sandblasting, for example, and have a surface roughness similar to a SPI/SPE surface roughness of C1, C2, C3, or D1, D2 or D3.

The disclosure still further provides methods that include introducing a guidewire as set forth herein into a patient, delivering a distal end of the guidewire to a target location, and performing a therapeutic or diagnostic function at the target location.

In some implementations, the method includes directing electrical energy to the distal tip of the guidewire to perform a tissue ablation function at the distal end of the guidewire. If desired, the method can further include directing a catheter over the guidewire to deliver a distal end of the catheter to the target location to perform a diagnostic and/or therapeutic function.

The disclosure also includes implementations of a method of transcatheter delivery of a device to the cardiovascular system. The method includes advancing a guidewire as described herein through a femoral vein to a venous crossing site, the venous crossing site being located along an iliac vein or the inferior vena cava. The method further includes using the guidewire to puncture a venous wall at the venous crossing site and then puncture an adjacent arterial wall at an arterial crossing site, the arterial crossing site being located along an iliac artery or the abdominal aorta, and advancing at least a portion of the guidewire into the iliac artery or the abdominal aorta, thereby forming an access tract between the venous crossing site and the arterial crossing site. The method further includes advancing a catheter through the access tract from the venous crossing site to the arterial crossing site, and delivering the device into the iliac artery or the abdominal aorta through the catheter.

In various implementations of the method, the device can be a prosthetic heart valve, an aortic endograft, a left ventricular assist device, or cardiopulmonary bypass device, for example. In various implementations, the guidewire can be selectively electrically energized to puncture the venous wall and the arterial wall. If desired, after delivering the device, the method can further include delivering an occlusion device over a guidewire into the access tract to close the access tract.

It is to be understood that both the foregoing general description and the following detailed description are exemplary and are intended to provide further explanation of the embodiments disclosed herein.

The accompanying drawings, which are incorporated in and constitute part of this specification, are included to illustrate and provide a further understanding of the method and system of the disclosure. Together with the description, the drawings serve to explain the principles of the disclosed embodiments.

### BRIEF DESCRIPTION OF THE DRAWINGS

The foregoing and other objects, aspects, features, and advantages of exemplary embodiments will become more apparent and may be better understood by referring to the following description taken in conjunction with the accompanying drawings, in which:

FIG. 1A depicts a side view of a core wire component of a guidewire in accordance with the present disclosure.

FIG. 1B is a schematic view of the core wire of FIG. 1A with a distal coil superimposed thereon showing placement of the distal coil.

### DETAILED DESCRIPTION

Reference will now be made in detail to the present preferred embodiments of the disclosure, examples of which are illustrated in the accompanying drawings. The method and corresponding steps of the disclosed embodiments will be described in conjunction with the detailed description of the system. The exemplary embodiments illustrated herein can be used to perform various procedures, but percutaneously. It will be appreciated, however, that the disclosed embodiments, or variations thereof, can be used for a multitude of procedures involving the connection of blood vessels or other biological lumens to native or artificial structures.

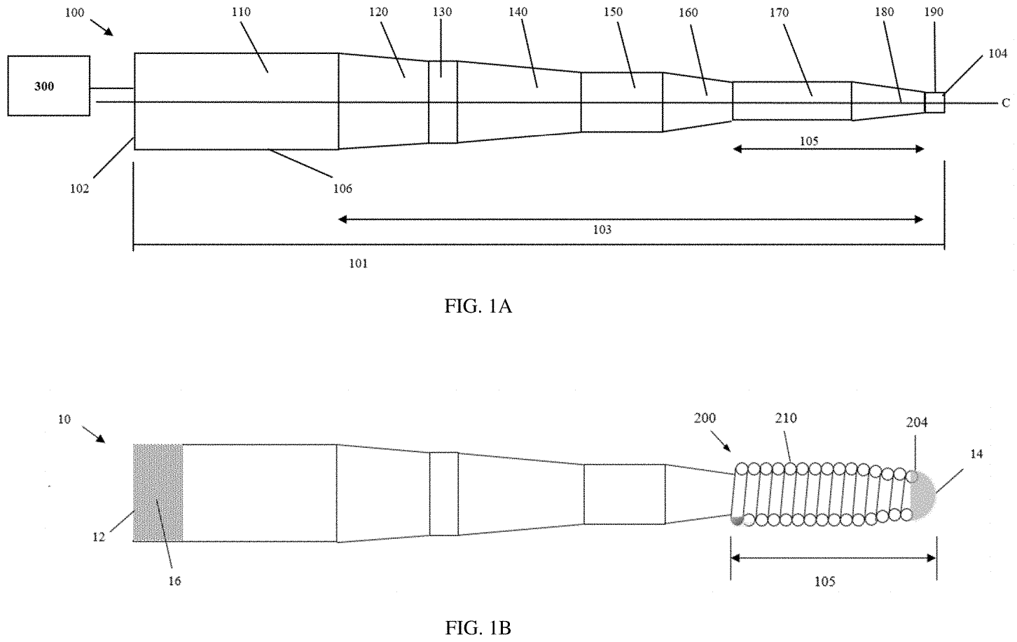

For purposes of illustration, and not limitation, FIG. 1A depicts a side view of a core wire component of a guidewire in accordance with the present disclosure, and FIG. 1B is a schematic view of the core wire of FIG. 1A with a distal coil superimposed thereon showing placement of the distal coil.

As illustrated in FIG. 1A, the core wire 100 is defined by an outer surface 106 between the proximal end 102 and the distal end 104 of the core wire. The core wire 100 has a centerline C that traverses the length of the core wire from the proximal end to the distal end of the core wire. The core wire includes a proximal region 100 having a first cross sectional dimension (e.g., 0.040 to about 0.028 inches, such as about 0.0320 inches) and a first length (e.g., 250-290 cm, such as about 270 cm) and a distal region 103 having a plurality of sections of different cross-sectional dimension 120, 130, 140, 150, 160, 170, 180, 190) that are smaller than the first cross-sectional dimension. The guidewire further includes a coil 200 wrapped around a distal end portion (170, 180, 190) of the core wire 100.

In some implementations, the distal region of the core wire can include at least three sections of different cross sectional dimension (e.g., 130, 150, 170) that are connected to each other by tapering transition regions (e.g., 120, 140, 160). A most proximal of the tapering transition regions 120 can be between about one and about two inches in length, or any increment therebetween of one tenth of an inch in length. A second most proximal of the tapering transition regions 140 can be between about three and about eight inches in length, or any increment therebetween of one tenth of an inch in length. A third most proximal of the tapering transition regions 160 can be between about one half and about one inch in length, or any increment therebetween of one tenth of an inch in length.

Sections of constant diameter of the distal region of the core wire can include, for example, a proximal most section 130 between about 0.2 and 1 inch in length, or any increment therebetween of one tenth of an inch in length and a width or diameter between 0.02 and about 0.03 inches in diameter (or any increment therebetween of about 0.001 inches in diameter), a second most proximal section 140 between about one and two inches in length, or any increment therebetween of one tenth of an inch in length, and a width or diameter between 0.008 and about. 010 inches in diameter (or any increment therebetween of about 0.001 inches in diameter) and a third most proximal section between about two and three inches in length, or any increment therebetween of one tenth of an inch in length and a width or diameter between 0.005 and about 0.008 inches in diameter (or any increment therebetween of about 0.001 inches in diameter). In other implementations, the distal region 105 of the core wire can include at least four sections of different cross sectional dimension (e.g., 130, 150, 170, 190) that are connected to each other by tapering transition regions (120, 140, 160, 180), and the lengths of the transition regions can be the same as or be similar to those set forth above. The distal most section 190 of the core wire can be about 0.003 and about 0.007 inches in diameter (or any increment therebetween of about 0.001 inches in diameter) and about 0.1 and about 0.5 inches in length, or any increment therebetween of one tenth of an inch in length, and the distal most tapering section can be between 0.2 and about 8 inches in length, or any increment therebetween of one tenth of an inch in length, and between 0.005 and about 0.009 inches in diameter or any increment therebetween of about 0.001 inches in diameter. The core wire 100 is preferably formed by grinding down a cylindrical starting material into the regions of progressively reduced diameter. Any desired thermal treatments can also be performed on the core wire after grinding to modify or optimize its mechanical properties.

In some embodiments, the distal end of the core wire can terminate in a section of constant diameter 190. If desired, the coil 200 can traverses between about 3 cm and about 12 cm of the length of the core wire, or any increment therebetween of one millimeter. The coil can traverse between about 5 cm and about 10 cm of the length of the core wire, or any increment therebetween of one millimeter.

In some embodiments, the core wire 100 can include a cobalt chromium alloy or other suitable material, such as 304 stainless steel. In some embodiments, the Co—Cr alloy can include carbon in a weight percent of 0.02 to 0.03 (e.g., 0.025), manganese in a weight percent of 0.10 to 0.20 (e.g., 0.15), silicon in a weight percent of 0.10 to 0.20 (e.g., 0.15), phosphorus in a weight percent of 0.010 to 0.020 (e.g., 0.015), sulfur in a weight percent of 0.005 to 0.020 (e.g., 0.01), chromium in a weight percent of 18-22 (e.g., 20 percent), nickel in a weight percent of 33-37 (e.g., 35 percent), molybdenum in a weight percent of 9-11 (e.g., 10 percent), titanium in a weight percent of 0.5-2 (e.g., 1 percent), iron in a weight percent of 0.5-2 (e.g., 1 percent), boron in a weight percent of 0.10-0.020 (e.g., 0.015 percent), with the balance being cobalt. The material can be heated and melted and re-solidified in order to enhance its mechanical properties. If desired, the coil 200 can include an alloy of platinum and tungsten to enhance radiopacity and mechanical properties.

The system further provides embodiments of an electrosurgical system that includes an electrical power source (e.g., 300) and a guide wire as described herein. The electrical power source 300 is configured to be selectively electrically coupled to the guide wire. Preferably, the coil 200 is welded to the core wire 200 (instead of by brazing, for example) to enhance its current carrying capacity and to reduce its propensity for melting when current is run through it in order to facilitate the delivery of electrical energy to a target tissue area.

The guidewire is preferably coated along a majority of its length with an electrically insulating material, such as a lubricious coating, such as PTFE (e.g., by dipping). Preferably, a proximal region or portion 16 of the guide wire (e.g., 0.5 to 1 inch) is exposed and not covered by the electrically insulating material. This proximal region of the core wire that is exposed can includes a roughened surface formed by sandblasting, for example, and have a surface roughness similar to a SPI/SPE surface roughness of C1, C2, C3, or D1, D2 or D3. In one implementation, the outer diameter of the distal coil 200 can be about 0.0112 inches. The entire structure including the core wire and the coil is then encased with a PTFE jacket to increase the overall diameter of the assembly to 0.014 inches. It is also possible to coat the guidewire core and coil with a ceramic or parylene coating, resulting, for example, in a coil 200 having a nominal diameter of 0.012 to 0.013 inches with a wall coating of about 0.0005-0.001 inches. But, it will be appreciated that these dimensions can be varied somewhat. For example, the grind profile illustrated in the FIGURES can be similar, but the maximum outer diameter of the proximal end of the guidewire can be between 0.010 and 0.020 inches, or any increment of 0.001 inches, with distal sections of the guidewire being of smaller relative diameter.

In a further implementation, the proximal end 12 of the guidewire 10 can be attached to a crimp (not shown) so other wires or sutures can be crimped thereto, if desired. This can be advantageous for electrified guidewires so as to avoid the need to exchange a shorter guidewire for a longer one to accommodate a catheter over its length. An adaptor can also be provided that connects the proximal end 12 of the guidewire to an electrosurgical generator. Preferably, the guidewire 10 is configured to be electrically coupled to a conventional electrosurgery generator such as the Medtronic Valleylab FX, which permits controlled actuation of a cutting switch that can be used to electrify the guidewire. In accordance with a preferred embodiment, the electrosurgical system is configured to permit a preset time-limit to individual actuations for each button press, such as 1 second timeout, before the button is again depressed. Preferably, the signal generator also has a switch lockout to assure no inadvertent actuation.

The disclosure still further provides methods that include introducing a guidewire as set forth herein into a patient, delivering a distal end of the guidewire to a target location, and performing a therapeutic or diagnostic function at the target location, such as crossing or cutting through the wall of a vessel or chamber.

In some implementations, the method includes directing electrical energy to the distal tip 14 of the guidewire 10 to perform a tissue ablation function at the distal end of the guidewire. If desired, the method can further include directing a catheter over the guidewire (not shown) to deliver a distal end of the catheter to the target location to perform a diagnostic and/or therapeutic function.

The disclosure also includes implementations of a method of transcatheter delivery of a device to the cardiovascular system, such as described in U.S. Pat. No. 10,058,315, which is incorporated by reference herein in its entirety for any purpose whatsoever. The method includes advancing a guidewire (e.g., 10) as described herein through a femoral vein to a venous crossing site, the venous crossing site being located along an iliac vein or the inferior vena cava. The method further includes using the guidewire to puncture a venous wall at the venous crossing site and then puncture an adjacent arterial wall at an arterial crossing site, the arterial crossing site being located along an iliac artery or the abdominal aorta, and advancing at least a portion of the guidewire into the iliac artery or the abdominal aorta, thereby forming an access tract between the venous crossing site and the arterial crossing site. The method further includes advancing a catheter through the access tract from the venous crossing site to the arterial crossing site, and delivering the device into the iliac artery or the abdominal aorta through the catheter as described in U.S. Pat. No. 10,058,315.

In various implementations of the method, the device can be a prosthetic heart valve, an aortic endograft, a left ventricular assist device, or cardiopulmonary bypass device, for example. In various implementations, the guidewire can be selectively electrically energized to puncture the venous wall and the arterial wall. If desired, after delivering the device, the method can further include delivering an occlusion device over a guidewire into the access tract to close the access tract.

The disclosed guidewires are particularly well suited for performing a Transcaval procedure as described in U.S. Pat. No. 10,058,315. Typical guidewire devices that have been used heretofore for this procedure are modified and used off-label for transcanal access. This off-label use is associated with complications and increased procedural times, as reported in Greenbaum et al (2014) where a significant (36%) number of subjects had multiple attempts at crossing. The specific tip design that is illustrated increases safety for the patient while crossing from the IVC to the abdominal aorta, for example. The table below highlights the key advantages of embodiments in accordance with the disclosure.

The disclosed embodiments can be expected to reduce procedure time and cost by eliminating the need for multiple wires. The proximal section of the guidewire 10 provides for controlled pushability of the wire during electrosurgical usage. The tapered transitions permit easier introductions of catheters and large bore introducers and guiding catheters over the guidewire. Thus, the disclosed guidewires can be used from the beginning until the end of such a procedure, including, for example, replacement of a heart valve with an artificial one during the procedure. The disclosed insulating jacket or layer reduces or eliminates unwanted electrical conductance and isolates energy delivery to just the tip of the guidewire. This isolated energy delivery combined with specifically design tip stiffness can reduce complications during the burning procedure, and can reduce wire prolapse and so-called “slit” burns.

In various embodiments herein, the disclosed guide wires can be provided with additional components found on other known guidewires, such as one or more nested coils surrounding the core wire, atraumatic distal ends, safety wires, and the like. Examples of such features can be found in one or more of U.S. Pat. Nos. 4,827,941, 5,617,875, 4,917,103, 4,922,923, 5,031,636 and U.S. Reissue Patent No. 34,466. Each of these patents is incorporated by reference herein in its entirety.

The devices and methods disclosed herein can be used for other procedures in an as-is condition, or can be modified as needed to suit the particular procedure. In view of the many possible embodiments to which the principles of this disclosure may be applied, it should be recognized that the illustrated embodiments are only preferred examples of the disclosure and should not be taken as limiting the scope of the disclosure.

What is claim is:

1. A guidewire, comprising:
a core wire having a proximal end, and a distal end and being defined by an outer surface between the proximal end and the distal end of the core wire, said core wire having a centerline that traverses the length of the core wire from the proximal end to the distal end of the core wire, the core wire including a proximal region having a first cross sectional dimension and a distal region having a plurality of sections of different cross-sectional dimension that are smaller than the first cross-sectional dimension; and
a coil wrapped around a distal end portion of the core wire, wherein the coil traverses between about 3 cm and about 12 cm of the length of the core wire.

2. The guidewire of claim 1, wherein the distal region of the core wire includes at least three sections of different cross sectional dimension that are connected to each other by tapering transition regions.

3. The guidewire of claim 1, wherein a most proximal of said tapering transition regions is between about one and about two inches in length.

4. The guidewire of claim 3, wherein a second most proximal of said tapering transition regions is between about three and about eight inches in length.

5. The guidewire of claim 3, wherein a third most proximal of said tapering transition regions is between about one half and about one inch in length.

6. The guidewire of claim 1, wherein the distal region of the core wire includes at least four sections of different cross sectional dimension that are connected to each other by tapering transition regions.

7. The guidewire of claim 1, wherein the distal end of the core wire terminates in a section of constant diameter.

8. The guidewire of claim 1, wherein the core wire includes a cobalt chromium alloy.

9. The guidewire of claim 1, wherein the coil includes an alloy of platinum and tungsten.

10. The guidewire of claim 1, wherein at least one of the core wire and the coil is coated with a layer of lubricious material.

11. An electrosurgical system comprising an electrical power source and a guide wire in accordance with claim 1, wherein the electrical power source is configured to be selectively electrically coupled to said guide wire.

12. The electrosurgical system of claim 11, wherein:
the coil is welded to the core wire to facilitate the delivery of electrical energy to a target tissue area;
the guidewire is coated along a majority of its length with an electrically insulating material;
a proximal region of the core wire is exposed and not covered by the electrically insulating material; and
the proximal region of the core wire that is exposed includes a roughened surface.

13. A method including introducing a guidewire according to claim 1 into a patient, delivering a distal end of the guidewire to a target location, and performing a therapeutic or diagnostic function at the target location.

14. The method of claim 13, further comprising directing electrical energy to the distal tip of the guidewire to perform a tissue ablation function at the distal end of the guidewire.

15. The method of claim 13, further comprising directing a catheter over the guidewire to deliver a distal end of the catheter to the target location.

16. A method of transcatheter delivery of a device to the cardiovascular system, comprising:
advancing a guidewire according to claim 1 through a femoral vein to a venous crossing site, the venous crossing site being located along an iliac vein or the inferior vena cava;
using the guidewire to puncture a venous wall at the venous crossing site and then puncture an adjacent arterial wall at an arterial crossing site, the arterial crossing site being located along an iliac artery or the abdominal aorta, and advancing at least a portion of the guidewire into the iliac artery or the abdominal aorta, thereby forming an access tract between the venous crossing site and the arterial crossing site;
advancing a catheter through the access tract from the venous crossing site to the arterial crossing site; and
delivering the device into the iliac artery or the abdominal aorta through the catheter.

17. The method of claim 16, wherein the device is a prosthetic heart valve, aortic endograft, left ventricular assist device, or cardiopulmonary bypass device.

18. The method of claim 16, wherein the guidewire is selectively electrically energized to puncture the venous wall and the arterial wall.

19. The method of claim 16, further comprising: after delivering the device, delivering an occlusion device over a guidewire into the access tract to close the access tract.
